(19) United States
(12) Patent Application Publication
(10) Pub. No.: US 2023/0145600 A1
(43) Pub. Date: May 11, 2023

(54) Non-invasive, Objective, Oculomotor, Vestibular, Reaction Time, and Cognitive Response Assessment Protocol for Post SARS Infection Based Neurological Injuries

(71) Applicant: Neurolign USA, LLC., Pittsburgh, PA (US)

(72) Inventor: Alexander D. Kiderman, Pittsburgh, PA (US)

(21) Appl. No.: 17/982,409

(22) Filed: Nov. 7, 2022

Related U.S. Application Data
(60) Provisional application No. 63/263,659, filed on Nov. 5, 2021.

(51) Int. Cl. A61B 5/00; A61B 3/02

(52) U.S. Cl. CPC A61B 5/4088; A61B 3/02; A61B 5/4023

(57) ABSTRACT

A method of assessing post SARS infection based neurological injuries using a quantitative, noninvasive, clinical objective, oculomotor, vestibular, reaction time, and cognitive response assessment protocol for evaluating post SARS infection based neurological injuries. The method comprises the steps of: coupling a VOG/VNG system to a subject wherein the VOG/VNG system is configured to present a plurality of Oculomotor, vestibular, reaction time, and cognitive tests to the subject; presenting a plurality of Oculomotor, vestibular, reaction time, and cognitive tests to the subject on the VOG/VNG system; obtaining objective physiologic responses of the patient from the plurality of Oculomotor, vestibular, reaction time, and cognitive tests to the subject via the VOG/VNG system; and using a plurality of the objective physiologic responses of the patient to assess post SARS infection based neurological injuries in the subject.

### RELATED APPLICATION

The present application claims the benefit of provisional patent application serial number 63/263,659 filed Nov. 5, 2022 which is incorporated herein by reference in its entirety.

### BACKGROUND OF THE INVENTION

### Field of the Invention

The present invention relates to testing for post-SARS infection based neurological injuries, and more specifically to quantitative, noninvasive, clinical objective, oculomotor, vestibular, reaction time, and cognitive response assessment protocol for post SARS infection based neurological injuries.

### Background Information

SARS-CoV-2 is the causative agent of COVID-19 which is highly contagious. Even with countermeasures taken globally to break the chain of transmission, as of Nov. 1, 2021, the number of people diagnosed with COVID-19 approached 250 million, including over 5 million deaths and as of November of 2022 the number of infections topped 600 million and the number of deaths was 6.5 million.

This enveloped positive-stranded RNA virus shows pathogenicity similar to that of another human coronavirus (HCoV). Individuals infected with SARS-COV-2 demonstrate a broad spectrum of clinical manifestations. Albeit primarily a disease of respiratory tract, the 2019 coronavirus infectious disease (COVID-19) has been found to have causal association with a plethora of neurological, neuropsychiatric and psychological effects. A SARS-CoV-2 (COVID-19) infection can, in fact, result in long-lasting neurological injuries, some of which may be unrecognized by the patient. See Maury A, Lyoubi A, Peiffer-Smadja N, de Broucker T, Meppiel E. Neurological manifestations associated with SARS-CoV-2 and other coronaviruses: A narrative review for clinicians. Rev Neurol (Paris). 2021 Jan-Feb;177(1-2):51-64. doi: 10.1016/j.neurol.2020.10.001. Epub 2020 Dec 16. PMID: 33446327; PMCID: PMC7832485; Roy D, Ghosh R, Dubey S, Dubey MJ, Benito-León J, Kanti Ray B. Neurological and Neuropsychiatric Impacts of COVID-19 Pandemic. Can J Neurol Sci. 2021 Jan;48(1):9-24. doi: 10.1017/cjn.2020.173. Epub 2020 Aug 5. PMID: 32753076; PMCID: PMC7533477; and Niazkar HR, Zibaee B, Nasimi A, Bahri N. The neurological manifestations of COVID-19: a review article. Neurol Sci. 2020 Jul;41(7):1667-1671. doi: 10.1007/s10072-020-04486-3. Epub 2020 Jun 1. PMID: 32483687; PMCID: PMC7262683.

Objective and accurate assessment of subtle neurological abnormalities can be difficult yet it is well understood that early and accurate detection of subtle neurological abnormalities is critical to identifying and managing long-term symptoms and deficits.

It is the object of the present invention to address the deficiencies of the prior art to yield quantitative, noninvasive, clinical objective, oculomotor, vestibular, reaction time, and cognitive response assessment protocol for post SARS infection based neurological injuries.

### SUMMARY OF THE INVENTION

The present invention is drawn to a method of assessing post SARS infection based neurological injuries using a quantitative, noninvasive, clinical objective, oculomotor, vestibular, reaction time, and cognitive response assessment protocol for evaluating post SARS infection based neurological injuries. The methodology will be applicable to other similar infection based neurological injuries other than SARS infections.

A method of assessing post SARS infection based neurological injuries in a subject comprises the steps of: coupling a VOG/VNG system to a subject wherein the VOG/VNG system is configured to present a plurality of Oculomotor, vestibular, reaction time, and cognitive tests to the subject; presenting a plurality of Oculomotor, vestibular, reaction time, and cognitive tests to the subject on the VOG/VNG system; obtaining objective physiologic responses of the patient from the plurality of Oculomotor, vestibular, reaction time, and cognitive tests to the subject via the VOG/VNG system; and using a plurality of the objective physiologic responses of the patient to assess post SARS infection based neurological injuries in the subject.

These and other advantages are described in the brief description of the preferred embodiments in which like reference numeral represent like elements throughout.

### BRIEF DESCRIPTION OF THE FIGURES

FIG. 1 is a schematic view of the dynamic vergence testing platform including 3d head mounted display system with integrated eye tracking technology for objective testing of vergence dysfunction for diagnosis and vergence recovery for convalescence;

FIG. 2 is a schematic view of the 3d head mounted display system of the vergence testing platform of FIG. 1;

FIG. 3 is a graph of the percentage of Abnormalities per OVERT-C Domain in the protocol of the present invention in a group of test subjects;

FIGS. 4A and 4B are comparison of test subjects NSI’s results with OVRT results according to the invention separating by severity of symptoms;

FIG. 5 illustrates a Univariate model of select parameters discrimination of post SARS infection based neurological injuries.

### DESCRIPTION OF THE PREFERRED EMBODIMENTS

It is noted that, as used in this specification and the appended claims, the singular forms “a,” “an,” and “the” include plural referents unless expressly and unequivocally limited to one referent. Within the following description the terms horizontal and vertical are relative to the conventional position of the subject’s eyes/vision, regardless of the subject’s actual head position, unless otherwise stated. Namely the subject’s eyes and the center of the subject’s vision will generally lie upon a horizontal plane (discounting variations in subject eye position for defining these reference directions). The vertical direction is perpendicular to the horizontal extending generally in the plane including the subject’s chin and the top of their head. Regarding to the subject invention, there is mounting evidence to support the theory that vergence dysfunction contributes to disability after mTBI. Similarly there is mounting evidence to support the theory that vergence recovery is an important aspect in mTBI convalescence.

Oculomotor, vestibular, reaction time, and cognitive (OVRT-C) assessments using eye-tracking techniques can provide objective measures of neural function. The protocol of this invention provides non-invasive tool that delivers a battery of OVRT-C tests to measure and quantify functional neurological abnormalities in people recovering from SARS-CoV-2. This evaluation will enhance clinical practice by providing novel and objective ways to screen for the long-term neurological impacts of SARS-CoV-2, as well as guiding rehabilitation strategies. Early identification of neural deficits caused by COVID-19 using the protocol of the present invention will help to better understand the neural substrates impacted by COVID-19 infection, aid in its management, and ideally treat or prevent its long-term consequences

The Oculomotor, vestibular, reaction time, and cognitive (OVRT-C) assessments is performed of a platform or system 100 which has been categorized as a type of Video-oculography (VOG) system 100. A suitable system is available from the applicant as the Neurolign Dx100™ system 100 which offers as lightweight frame with a soft gasket for snug, light-free fit (595 grams/ 1 lb. 5 oz.), Dioptric Adjustment from +4 to -4, Inter-Pupillary Distance (IPD) adjustment, Adjustable head strap, a Flexible housing designed to fit 5th to 95th percentile of patients. Further details of the preferred Neurolign Dx100™ system 100 include Field of View: 60°, Display Resolution: 2560 x 1440 pixel, 3D image perception, Synchronization between eye tracking and stimulus, Sample rate: 100-1000 frames per second (100-1000 Hz), Eye tracking range: ±30 degrees horizontal, ±30 degrees vertical, ±10 degrees torsional, and pupil area, Spatial eye-tracking resolution: horizontal, vertical, and torsion <0.1 degrees, and Infrared Illumination: 940 nm (infrared), continuous, near frontal illumination. The preferred Neurolign Dx100™ system 100 has an advantage over other testing platforms that it tests oculomotor and vergence function in an entirely virtual environment.

More generally, as background, VOG systems have been defined by Richard E. Gans, PhD, who is the Founder and Executive Director of the American Institute of Balance and he served on the board of the American Academy of Audiology, in the Hearing Journal: May 2001 - Volume 54 - Issue 5 - pp 40, 42 “Video-oculography is a method of recording eye movement through the use of digital video cameras. This is a significant change from electronystagmography, which uses the corneal retinal potential, which is the eye’s battery-like effect. As the eyes move side to side and up and down, the corneo-retinal potential’s positive and negative discharge is recorded. VOG technology, however, uses infrared cameras to measure the eye’s position. Small cameras, mounted in goggles, track the center of the pupil to provide the location of the eye.”

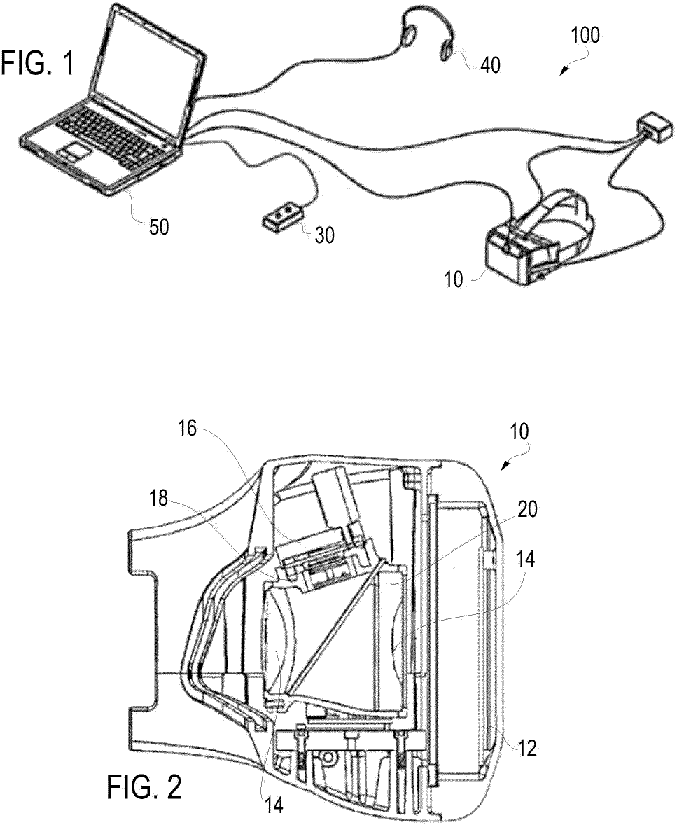

FIG. 1 is a schematic view of the system 100 for performing quantitative, noninvasive, clinical objective, oculomotor, vestibular, reaction time, and cognitive response assessment protocol for post SARS infection based neurological injuries according to the invention. The system 100 includes a 3d head mounted display system 100 with integrated eye tracking technology. The system 100 includes the head mounted goggle unit 10, user input device 30, headphones 40 for auditory input for instructions or stimulus and/or subject isolation, coupled to a laptop 50 to yield a highly portable system. FIG. 2 is a schematic design of head mounted VOG/VNG goggle unit 10 with OLED micro display or VR screen 12, two sets of optics 14, cameras 16 for recording eye movement typically at 100-1000 hz, micro LEDs 18 for illumination of the eyes, and a hot mirror. Simply, the VR screen 12 provides the visual stimulus and the cameras 16 capture eye response for quick analysis. The details of the VR display screen 12 are believed to be known to those or ordinary skill in the art and it allows the system 100 to present visual images or targets to the user that have a perceived or simulated distance for vergence testing. The eye tracking technology described herein is generally known in the art.

The VOG/VNG system 100 is coupled to the subject and configured to present a plurality of Oculomotor, vestibular, reaction time, and cognitive (OVRT-C) tests to the subject in accordance with the protocol discussed below. The system 100 is designed to non-invasively obtain objective physiologic responses of the subject from the eye tracking unit based upon the Oculomotor, vestibular, reaction time, and cognitive (OVRT-C) tests presented to the subject. The combination of the eye tracking and the display of simulated distanced visual targets allow the VOG/VNG system 100 to automatically run a number of preprogrammed neurologic Oculomotor, vestibular, reaction time, and cognitive (OVRT-C) tests and to record the physiologic responses thereto. The specific protocol and key output variables in accordance with one aspect of the invention is shown below

| | |
|---|---|
| Test | Key Output Variables |
| Spontaneous Nystagmus | PSFV for horizontal and vertical nystagmus with fixation and in the dark |
| Gaze Horizontal | PSFV for horizontal and vertical nystagmus with fixation and in the dark |
| Horizontal Random Saccade | Left/Rightward eye velocity, latency, accuracy and final accuracy. |
| Vertical Random Saccade | Up/Down eye velocity, latency, accuracy and final accuracy. |
| Predictive Saccade | First predicted, total predicted, Predicted % |
| Smooth Pursuit Horizontal, .1 Hz | Left/Rightward gain, saccadic components, # of square jerks |
| Smooth Pursuit Horizontal, 0.75 Hz | Left/Rightward gain, saccadic components, # of square jerks |
| Smooth Pursuit Vertical, .1 Hz | Up/Down gain, saccadic components, # of square jerks |
| Smooth Pursuit Vertical, .75 Hz | Up/Down gain, saccadic components, # of square jerks |
| Vergence Pursuit | Left/Right eyes near and far angle and symmetry |
| Self-Paced Saccade | Saccade per second, eye velocity and inter-wall consistency |
| Anti-saccade | Left/Rightward eye velocity, latency and error rate |
| Optokinetic Horizontal and 20 & 60 d/s | Average gain and asymmetry for slow phase, area under fit for fast phase |
| Auditory Reaction Time | Left/Right button’s latency and SD |
| Visual Reaction Time | Left/Right button’s latency and SD |
| Saccade and Reaction Time | Saccade response: Left/rightward eye velocity, latency, accuracy and final accuracy. Motor response: Left/right button latency and SD |
| Light reflex | Constriction velocity |
| Subjective Visual Vertical | Error, deg |
| Subjective Visual Horizontal | Error, deg |

### Spontaneous Nystagmus Testing

Nystagmus is generally defined as any involuntary, rapid, rhythmic eye movement (horizontal, vertical, rotatory, or combinations thereof) and spontaneous nystagmus is generally defines as that nystagmus occurring without specific stimulation of the vestibular system. The system 100 obtains horizontal and vertical nystagmus variables in a spontaneous nystagmus test both with a central (origin) fixation light and in the dark. A fixation light can suppress a subject’s nystagmus response. The system 100 calculates Peak slow phase velocity (PSVF) for horizontal and vertical eye movement components with and without the target or fixation light on. PSFV is a considered a key variable for the evaluating the subjects in the present protocol.

### Gaze Horizontal Testing

In Gaze Horizontal (GH) testing the subject is directed to fixate on a target light or stimulus for 3 seconds, which target is located 10° to the right of an origin or center position. The stimulus or Light is then extinguished for 15 seconds. The subject is directed to fixate on a target light for 3 seconds, which is located 10° to the left of center. Light is then extinguished for 15 seconds. The unit 10 will calculate Peak slow phase velocity (PSFV) for horizontal and vertical eye movement components with and without the target or fixation light on. PSFV is a considered a key variable for the evaluating the subjects in the present protocol. The GH testing may provide values separately for the left and right eyes.

### Horizontal and Vertical Random Saccade Testing

In the Horizontal random saccade (HS) the subject is directed to follow (or “jump” to) a target (the stimulus, such as a dot, although other stimulus may be utilized) as it is displayed at a fixed location on the screen. The visual stimulus is presented in this test at pseudo-randomly distributed times (between 1 to 2 seconds) and will exhibit displacements from -30 to +30 degrees measured along the horizontal axis for the horizontal testing. A number of trials, or saccadic movements, will be observed. The unit 10 will obtain values for at least eye peak velocity, latency, accuracy for both main saccade, and combined main and secondary corrective saccade. Each variable is calculated separately for left and right eyes. Vertical saccade (VS) is analogous to Horizontal random saccade but in a vertical orientation for stimulus movement, and the range of movement is commensurate with vertical eye range (displacements from -20 to +20 degrees along the vertical axis).

### Predictive Saccade Testing

In the predictive saccade testing, also called Horizontal predictive saccade (HPS) testing as this is performed on the horizontal axis for this testing, the subject is directed to view a visual stimulus as it is quickly displayed at a fixed location. Subject will be presented with 6 pseudo-random saccade stimuli followed by 20 “mirrored” saccade stimuli meaning these have a repeated displacement of +/-10 degrees in the horizontal direction and the stimuli are presented at a constant time interval of 0.65 seconds. In the HPS testing the unit 10 will measure the first predicted saccade which is the number of mirrored saccade stimuli until the main latency is less than zero for that stimuli trial, although a number slightly higher than zero (i.e. a minimal threshold latency) could be used as such could still be indicative of a predictive aspect if the number of threshold is small enough.

Most subjects will reach a predictive saccade within several repetitions. In the HPS testing the unit 10 will measure the percentage of predicted saccades which is merely the number of saccadic stimuli having the latency lower than zero (or a slightly higher threshold than zero, if desired) divided by the total number of mirrored stimulus. As noted from this description, in the HPS testing the unit 10 will measure the main saccade latency. Each variable may be calculated separately for left and right eyes in HPS testing.

### Horizontal and Vertical Smooth Pursuit Testing

In the Smooth pursuit horizontal (SPH) testing the subject is directed to follow a visual stimulus (e.g. a dot on screen 12) as it moves through a sinusoidal displacement of +/-10 degrees along the horizontal axis. The SPH testing is run at two distinct frequencies, namely at 0.1 Hz and at 0.75 Hz.

In SPH testing the unit 10 measures the Velocity gain (also called the pursuit gain) to the right and to the left. The velocity gain is ratio of the eye velocity to the target velocity. In SPH testing the unit 10 measures the velocity gain asymmetry which is the difference between the gain to the right and to the left. In SPH testing the unit 10 measures velocity phase to the right and to the left which is a measure of the patient eye velocity relative to the target velocity profile. In SPH testing the unit 10 measures the percent of saccade, which is the percent of saccadic eye movement components that comprise the whole of the smooth pursuit test. In SPH testing the unit 10 measures the position gain to the right and to the left which is comparison of the eye position to the target position and asymmetry values between right and left. In SPH testing the unit 10 provides a spectral purity measurement and an initiation latency measurement. The SPH testing may provide values separately for the left and right eyes.

In Smooth pursuit vertical (SPV) testing the subject is directed to follow a target as it moves through a sinusoidal displacement of +/-10 degrees along the vertical axis, analogous to the SPH testing discussed above.

### Vergence Pursuit Testing

In vergence pursuit testing the device 100 will present a continuously, smoothly transitioning movement of the stimuli, creating the appearance of a target gradually moving toward or away from the subject in the virtual depth space. This will encourage subjects to make continually updated, smoothly transitioning convergence and divergence eye movements. For the vergence smooth pursuit test, subjects visualized the stimulus moving towards and away in a sinusoidal pattern at 0.1 Hz. The following variables were determined to be key variables for analysis, namely Near and far angle (measures of the angle of the left and right eye with the target at the nearest point and the farthest point, respectively, in its sinusoidal movement), Excursion (a measure of the difference between the near and far angle, or an amplitude measurement), Lag time (a measure of the delay between target movement and tracking eye movement) and Symmetry (a measure of the comparison of the left and the right eye movements).

For Vergence Pursuit testing, data will be both segmented into individual cycles (sub-segments of the target movement profile, e.g., cycles of a sinusoidal-modulated stimulus) and analyzed per cycle, or analyzed for the whole test. The following are examples of measures that will be generated by the method or device for Vergence Pursuit testing, both per cycle and for the whole test: The correlation between the movements of the two eyes during target presentation; The lag (temporal shift) of the eye movement relative to the virtual position of the stimulus; The amplitude or gain of the eye position relative to the virtual position of the stimulus at any or all time points during the test; The presence and amount of saccadic movement during the test; and The asymmetry, between the two eyes, of any of the previous three measures (saccades, lag, gain).

### Self Paced Saccade Testing

Self-Paced Saccade testing is measurements of a subjects voluntary saccades made between two stationary targets in a fixed period of time. The system will evaluate saccades per second, eye velocity and inter-eye consistency.

### Optokinetic Testing

In the two Optokinetic (OKN) tests the patients will see stimulus (e.g., lighted dots moving on the display first to the right, then to the left. The two optokinetic stimulus will be at rotation rates or speeds of 20 and 60 deg/sec, respectively. Each test consists of 15 seconds clockwise (CW) and 15 seconds counterclockwise (CCW) rotation stimulus. The unit 10 will measure at least the Average slow phase gain, average slow phase asymmetry, fast phase velocity vs. amplitude, and fast phase velocity asymmetry for each test and for left and right eyes.

### Visual and Auditory Reaction Time Testing

In the Visual reaction time (VRT) test randomly timed center lights or stimulus are presented. The subject is directed to signal their recognition by pressing a button, or other input. The system 100 measures the Average visual reaction time and the standard deviation (SD) of the reaction time. In the Auditory reaction time (ART) test randomly timed pulses of sound are presented to the subject through an associated audio output (speaker) and the subject is directed to signal their recognition by pressing a button. The system 100 measures the Average audio reaction time (latency) and the SD of the reaction time.

### Saccade Reaction Time Testing

In the Saccade and reaction time (SVRT) test visual saccadic stimuli are randomly projected from 1 to 2 seconds and displacement of -30 to +30 degrees. The patient is directed to gaze at the saccadic stimulus and then also press either the left or right button (or other input device) to record whether the stimulus was projected to the right or to the left. The unit 10 measures the same descriptive variables as regular saccade (HS and VS) along with latency, SD, and percent of error for each direction.

### Light Reflex Testing

In Light Reflex (LR) testing a central stimulus (e.g. a light spot or dot) is projected for 300 milliseconds and extinguished for 3 seconds and the sequence will be repeated 10 times. The system 100 measures pupil reaction latency, constriction velocity, and amplitude separately for the left and right eyes.

### Subjective Visual Vertical and Horizontal Testing

In the subjective visual vertical (SVV) testing the subject is presented with a red line on the display and directed to use control left and right buttons (or any desired input device such as a joystick etc.) to manipulate the displayed line into the vertical (upright). One input button rotates the line in one direction and the other input device rotates the line in the other. Subject is directed to inform the clinician when they are finished and they believe the line is vertical, known as the subjective vertical position. The unit 10 measures the mean and standard deviation from subjective vertical position and the true vertical position.

In the subjective visual Horizontal (SVH) testing the subject is presented with a red line on the display and directed to use control left and right buttons (or any desired input device such as a joystick etc.) to manipulate the displayed line into the horizontal (flat). One input button rotates the line in one direction and the other input device rotates the line in the other. Subject again is directed to inform the clinician when they are finished and they now believe the line is horizontal, known as the subjective horizontal position. The system 100 measures the mean and standard deviation from subjective horizontal position and the true horizontal position.

### OVRT Norm Database

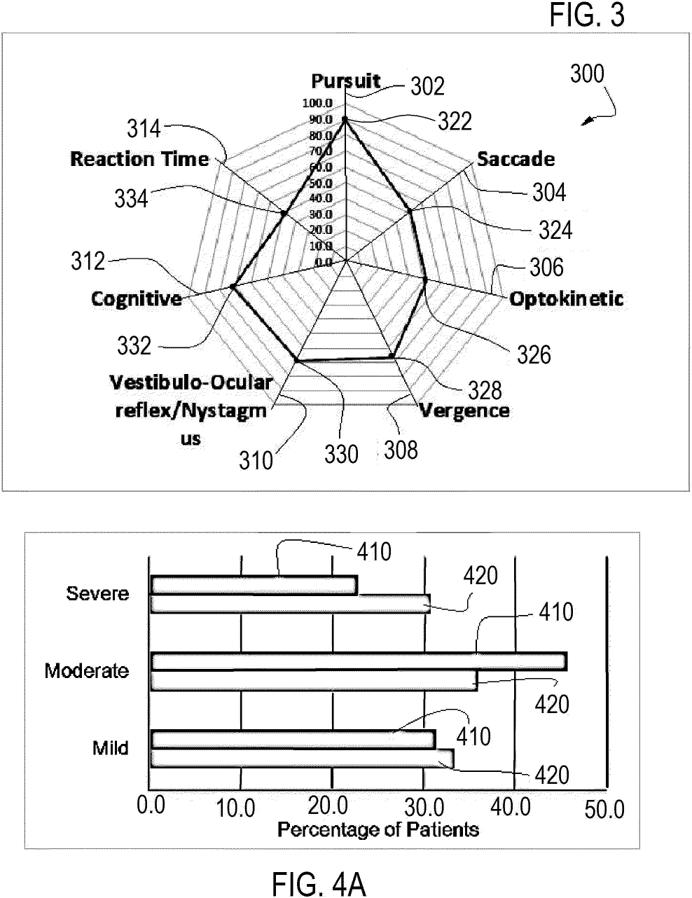

The results of each of the above tests are compared with an FDA-approved OVRT norms database of known healthy adults aged 18-45 years. Covid-19 Recovered patients’ data from the protocol of the present invention was analyzed and percentage of abnormal responses were calculated and the results in each of these domains is shown in FIG. 3. FIG. 3 is a graph 300 of the percentage of Abnormalities per OVERT-C Domain in the protocol of the present invention in a group of test subjects. The domains are pursuit 302, saccade 304, optokinetic 306, vergence 308, Vestibular Ocular reflex 310, cognitive 312, and reaction time 314, with each having its own “axis” in graph 300. Abnormal responses are those outside of accepted thresholds for the given parameter based upon the FDA-approved OVRT norms database of known healthy adults aged 18-45 years. Graph 300 illustrates the percentage of Abnormalities in the protocol of the present invention in a group of test subjects for pursuit 322, saccade 324, optokinetic 326, vergence 328, Vestibular Ocular reflex 330, cognitive 332, and reaction time 334.

### Neurobehavioral Symptom Inventory

The Neurobehavioral Symptom Inventory (NSI) is a 22-item self-report measure of symptoms, Scale 0 (none) to 4 (severe). The NSI was developed to subjectively measure post-concussion symptoms following traumatic brain injury. Although a subjective guide the NSI has demonstrated high internal consistency and studies suggest that the NSI is a reliable and valid measure of post-concussive symptoms. NSI has not been limited to TBI and extends, as here, to subjective evaluation neurological disorders.

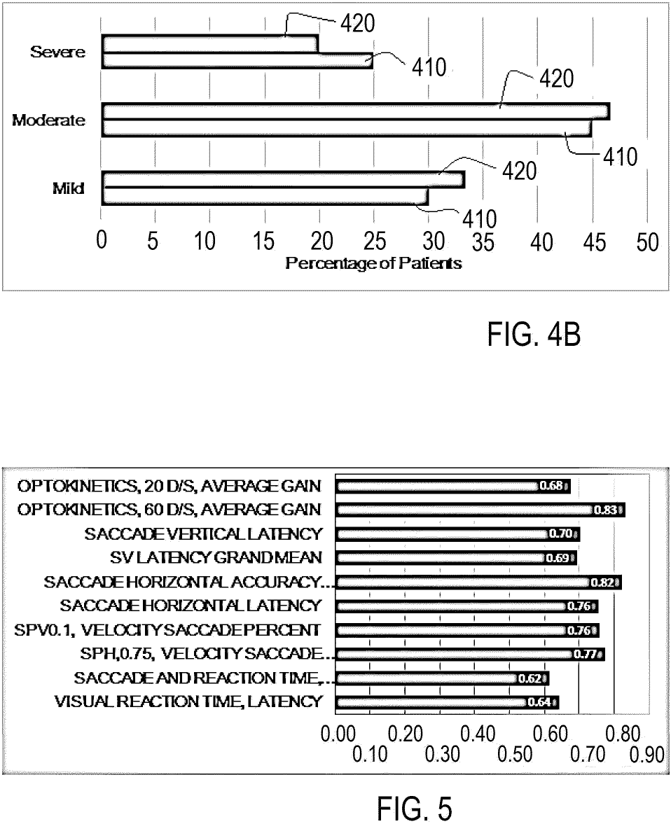

Supplementary to OVRT-C tests, Neurobehavioral Symptom Inventory (NSI) information were collected for test subject of the protocol of the present invention. The NSI results 410 and protocol results 420 of two sets of subjects were compared, with the NSI results 410 and the protocol results 420 used to divide the subjects into Mild, Moderate and Severe groupings. The Mild group was considered to be a <16 NSI score and a <20 OVRT-C percentage of abnormalities in the present protocol. The Moderate group was considered to be a 16-30 NSI score and a 20-50 OVRT-C percentage of abnormalities in the present protocol. The Severe group was considered to be >30 NSI score and >50 OVRT-C percentage of abnormalities in the present protocol. FIGS. 4A and 4B are comparison of test subjects NSI’s results 410 with OVRT results 420 according to the invention separating by severity of symptoms. In FIG. 4A the NSI results are on top and the OVRT-C results of the protocol on the bottom, while in FIG. 4B the NSI results are on bottom and the OVRT-C results of the protocol on the top. The results show a close correlation and the protocol of the invention can be used to detect the presence of abnormalities as well as accurate assess the severity thereof.

### Predictive Variables

The protocol of the present invention need not evaluate every parameter of every test for effectiveness. FIG. 5 shows a logistics regression, univariate model (Area under receiver operating characteristics) for select variables as an individual variable discriminating neurologic dysfunction in test subjects. The combination of any four of these characteristics together leads to an AUC>0.9, or extremely discriminating (accurate).

It is understood, therefore, that this invention is not limited to the particular embodiments disclosed, but it is intended to cover modifications that are within the spirit and scope of the invention, as defined by the appended claims and equivalents thereto. The preferred embodiments described above are illustrative of the present invention and not restrictive hereof. It will be obvious that various changes may be made to the present invention without departing from the spirit and scope of the present invention. The precise scope of the present invention is defined by the appended claims and equivalents thereto.

What is claimed is:

1. A method of assessing post SARS infection based neurological injuries within a subject using a quantitative, noninvasive, clinical objective, oculomotor, vestibular, reaction time, and cognitive response assessment protocol for evaluating post SARS infection based neurological injuries.

2. A method of assessing post SARS infection based neurological injuries in a subject comprising the steps of:
coupling a VOG/VNG system to a subject wherein the VOG/VNG system is configured to present a plurality of Oculomotor, vestibular, reaction time, and cognitive tests to the subject;
presenting a plurality of Oculomotor, vestibular, reaction time, and cognitive tests to the subject on the VOG/VNG system;
obtaining objective physiologic responses of the patient from the plurality of Oculomotor, vestibular, reaction time, and cognitive tests to the subject via the VOG/VNG system; and
using a plurality of the objective physiologic responses of the patient to assess post SARS infection based neurological injuries in the subject.

3. The method of assessing post SARS infection based neurological injuries in a subject according to claim 2 wherein the step of using a plurality of the objective physiologic responses of the patient to assess post SARS infection based neurological injuries in the subject includes a comparison of a plurality of the objective physiologic responses of the patient an FDA-approved OVRT norms database of physiologic responses of known healthy adults.

4. The method of assessing post SARS infection based neurological injuries in a subject according to claim 2 further including the step of performing a logistics regression, univariate model for the objective physiologic responses of the patient from the plurality of Oculomotor, vestibular, reaction time, and cognitive tests to the subject via the VOG/VNG system to identify the plurality of the objective physiologic responses of the patient used to assess post SARS infection based neurological injuries in the subject.

5. The method of assessing post SARS infection based neurological injuries in a subject according to claim 4 wherein the plurality of the objective physiologic responses of the patient used to assess post SARS infection based neurological injuries in the subject together have an AUC>0.9.

6. The method of assessing post SARS infection based neurological injuries in a subject according to claim 2 wherein the assessment of the post SARS infection based neurological injuries in the subject includes a categorization the subject as one of Mild, Moderate and Severe grouping.

7. The method of assessing post SARS infection based neurological injuries in a subject according to claim 2 wherein the step of obtaining objective physiologic responses of the patient from the plurality of Oculomotor, vestibular, reaction time, and cognitive tests to the subject via the VOG/VNG system includes physiologic responses of the patient for at least pursuit, saccade, optokinetic, vergence, Vestibular Ocular reflex, cognitive, and reaction time domains.

8. The method of assessing post SARS infection based neurological injuries in a subject according to claim 2 wherein the VOG/VNG system is configured to automatically run a number of preprogrammed neurologic Oculomotor, vestibular, reaction time, and cognitive tests and to record the physiologic responses thereto.

9. The method of assessing post SARS infection based neurological injuries in a subject according to claim 2 wherein the step of presenting a plurality of Oculomotor, vestibular, reaction time, and cognitive tests to the subject on the VOG/VNG system includes spontaneous nystagmus testing.

10. The method of assessing post SARS infection based neurological injuries in a subject according to claim 2 wherein the step of presenting a plurality of Oculomotor, vestibular, reaction time, and cognitive tests to the subject on the VOG/VNG system includes Gaze Horizontal testing.

11. The method of assessing post SARS infection based neurological injuries in a subject according to claim 2 wherein the step of presenting a plurality of Oculomotor, vestibular, reaction time, and cognitive tests to the subject on the VOG/VNG system includes Horizontal random saccade and Vertical random saccade.

12. The method of assessing post SARS infection based neurological injuries in a subject according to claim 2 wherein the step of presenting a plurality of Oculomotor, vestibular, reaction time, and cognitive tests to the subject on the VOG/VNG system includes predictive saccade testing.

13. The method of assessing post SARS infection based neurological injuries in a subject according to claim 12 wherein predictive saccade testing will measure the first predicted saccade wherein the the main latency is less than a minimal threshold latency.

14. The method of assessing post SARS infection based neurological injuries in a subject according to claim 2 wherein the step of presenting a plurality of Oculomotor, vestibular, reaction time, and cognitive tests to the subject on the VOG/VNG system includes horizontal and vertical smooth pursuit testing.

15. The method of assessing post SARS infection based neurological injuries in a subject according to claim 2 wherein the step of presenting a plurality of Oculomotor, vestibular, reaction time, and cognitive tests to the subject on the VOG/VNG system includes vergence pursuit testing.

16. The method of assessing post SARS infection based neurological injuries in a subject according to claim 2 wherein the step of presenting a plurality of Oculomotor, vestibular, reaction time, and cognitive tests to the subject on the VOG/VNG system includes self paced saccade testing and saccade reaction time testing.

17. The method of assessing post SARS infection based neurological injuries in a subject according to claim 2 wherein the step of presenting a plurality of Oculomotor, vestibular, reaction time, and cognitive tests to the subject on the VOG/VNG system includes optokinetic testing.

18. The method of assessing post SARS infection based neurological injuries in a subject according to claim 2 wherein the step of presenting a plurality of Oculomotor, vestibular, reaction time, and cognitive tests to the subject on the VOG/VNG system includes visual and auditory reaction time testing.

19. The method of assessing post SARS infection based neurological injuries in a subject according to claim 2 wherein the step of presenting a plurality of Oculomotor, vestibular, reaction time, and cognitive tests to the subject on the VOG/VNG system includes light reflex testing.

20. The method of assessing post SARS infection based neurological injuries in a subject according to claim 2 wherein the step of presenting a plurality of Oculomotor, vestibular, reaction time, and cognitive tests to the subject on the VOG/VNG system includes subjective visual vertical and horizontal testing.
